(19) United States
(12) Patent Application Publication
(10) Pub. No.: US 2024/0325011 A1
(43) Pub. Date: Oct. 3, 2024

(54) VASCULAR CLOSURE SYSTEM WITH INTRODUCER FOR SHEATH TRANSFER

(71) Applicant: Teleflex Life Sciences LLC, Wilmington, DE (US)

(72) Inventors: Greg A. WALTERS, Exton, PA (US); Joseph Todd GRINTZ, Glenmoore, PA (US)

(73) Assignee: Teleflex Life Sciences LLC, Wilmington, DE (US)

(21) Appl. No.: 18/742,624

(22) Filed: Jun. 13, 2024

Related U.S. Application Data
(63) Continuation of application No. 16/077,525, filed on Aug. 13, 2018.
(60) Provisional application No. 62/278,298, filed on Jan. 13, 2016.

(51) Int. Cl. A61B 17/00; A61M 25/06; A61M 25/09

(52) U.S. Cl. CPC A61B 17/0057; A61M 25/06; A61M 25/09; A61B 2017/00659; A61B 2017/00672

(57) ABSTRACT

A system includes an introducer configured to be inserted through the puncture along a guidewire that extends through the puncture into the vessel. The introducer includes an introducer body, a proximal tapered tip, a distal tapered tip that is opposite to the proximal tapered tip along the central axis, and a bore that extends from the proximal tapered tip to the distal tapered tip along the central axis. The system includes an access sheath configured to be inserted over the introducer. The access sheath includes a front end, a rear end opposite to the front end, and a lumen that extends from the front end to the rear end. The proximal tapered tip of the introducer is configured to be inserted into the front end and through the lumen of access sheath, such that a movable interference fit is attained between the access sheath and the introducer.

### CROSS-REFERENCE TO RELATED APPLICATIONS

This application is a continuation of U.S. patent application Ser. No. 16/077,525, filed Aug. 13, 2018, which is a national phase application of PCT/US2017/013314, filed Jan. 13, 2017, which claims the benefit and priority to U.S. Provisional Application Ser. No. 62/278,298, filed Jan. 13, 2016, the entire disclosures of which are incorporated by reference into the present application for all purposes.

### TECHNICAL FIELD

The present disclosure relates to vascular closure system with an introducer configured for sheath transfer.

### BACKGROUND

During an interventional cardiovascular procedure a puncture may be made in the femoral artery. Advanced cardiovascular procedures may obtain access to the aorta via the vena cava in situations where the femoral artery is not a suitable approach path. In one example, the procedure is a trans-caval aortic valve replacement procedure, or “trans-caval” procedure. Vascular closure devices composed of an absorbable intra-arterial toggle, an extra-vascular folding scaling plug, and a connecting suture, such as a filament, have been developed and may be used to seal these punctures. These devices function by compressing the intra and extra-arterial components together around the puncture, with sufficient tension within the connecting suture. However, as the size of percutaneous sheaths become larger to accommodate larger cardiovascular devices, the size of the resulting puncture increases. Larger punctures are harder to seal because of the larger vessel wall defect or puncture. In the case of sealing blood pressure with an external plug, larger defects expose the plug to increased forces, which must be supported through the connecting suture by the intra-arterial toggle.

### SUMMARY

An embodiment of the present disclosure is a system configured to seal a puncture in the vessel. The system includes an introducer configured to be inserted through the puncture along a guidewire that extends through the puncture into the vessel. The introducer includes an introducer body that is elongate along a central axis, a proximal tapered tip, a distal tapered tip that is opposite to the proximal tapered tip along the central axis, and a bore that extends from the proximal tapered tip to the distal tapered tip along the central axis. The bore is sized to receive the guidewire therethrough, wherein the distal tapered tip is configured to be inserted into the vessel along the guidewire. The system includes an access sheath configured to be inserted over the introducer and into the vessel. The access sheath includes a front end, a rear end opposite to the front end, and a lumen that extends from the front end to the rear end. The proximal tapered tip of the introducer is configured to be inserted into the front end and through the lumen of the access sheath, such that a movable interference fit is attained between the access sheath and the introducer. The system further includes a vascular closure device including a sealing element configured to seal the puncture of the vessel. When the introducer has been removed from the lumen of the access sheath, the rear end of the access sheath is configured to receive the vascular closure device such that the sealing element extends out of the front end of the access sheath. Related methods are disclosed.

The use of the introducer limits blood loss during an exchange of a procedure access sheath used to guide a catheter (or other medical device) into the vessel, e.g. the femoral artery or the aorta, with an access sheath for use with a vascular closure device.

### BRIEF DESCRIPTION OF THE DRAWINGS

The foregoing summary, as well as the following detailed description of an exemplary embodiments of the application, is better understood when read in conjunction with the appended figures. The figures illustrate exemplary embodiments for the purposes of illustration. It should be understood, however, that the application is not limited to the precise arrangements and systems shown.

FIG. 1 is a perspective view of a vascular closure system in accordance with an embodiment of the present disclosure;

FIG. 2A is a perspective view of an introducer and an access sheath of the system shown in FIG. 1;

FIG. 2B is a perspective view of the vascular closure device and access sheath of the system shown in FIG. 1;

FIG. 3 is a side view of the introducer shown in FIG. 1;

FIG. 4A is a side view an introducers according to an embodiment of the present disclosure;

FIG. 4B is a side view an introducers according to another embodiment of the present disclosure;

FIG. 4C is a side view an introducers according to another embodiment of the present disclosure;

FIG. 5A is a perspective view of a vascular closure device in accordance with an embodiment of the present disclosure;

FIG. 5B is a partial cut-away view of the vascular closure device shown in FIG. 5A;

FIG. 5C is a perspective view of a sealing device associated with the vascular closure device in FIG. 5A;

FIG. 5D is a side sectional view of a distal portion of the vascular closure device;

FIGS. 6A-6C are rear perspective views of the vascular closure device with portions of the device removed for clarity;

FIG. 6D is a sectional view of the vascular closure device shown in FIG. 6A;

FIG. 6E is a perspective view of the release component, delivery component, and tensioner of the vascular closure device shown in FIG. 6A;

FIG. 6F is a perspective cross-sectional view of the a release component, delivery component, and a tensioner of the vascular closure device shown in FIG. 6E, taken along line 6F-6F;

FIG. 6G is a perspective view of the delivery component and tensioner of the vascular closure device shown in FIG. 6E;

FIGS. 6H and 6I are perspective and top views, respectively, of the release component of the vascular closure device shown in FIG. 5A;

FIG. 7A is a schematic showing an access sheath from a cardiovascular surgical procedure partially disposed within a vessel through puncture sites in a vessel;

FIG. 7B is a schematic showing the introducer from FIG. 1 inserted in the access sheath shown in FIG. 7A;

FIG. 7C is a schematic showing the removal of the access sheath from the vessel with the introducer remaining in position;

FIG. 7D is a schematic showing the access sheath of the system shown in FIG. 1 inserted into the vessel along the introducer sheath;

FIG. 7E is a schematic showing the introducer removed from the access sheath and the vascular closure device positioned for insertion into the access sheath;

FIG. 7F is a schematic showing the sealing element fully sealing the puncture site.

### DETAILED DESCRIPTION OF ILLUSTRATIVE EMBODIMENTS

Certain terminology is used in the following description for convenience only and is not limiting. The words “right”, “left”, “lower” and “upper” designate directions in the drawings to which reference is made. The words “proximally” and “distally” refer to directions toward and away from, respectively, the individual operating the system. The terminology includes the above-listed words, derivatives thereof and words of similar import.

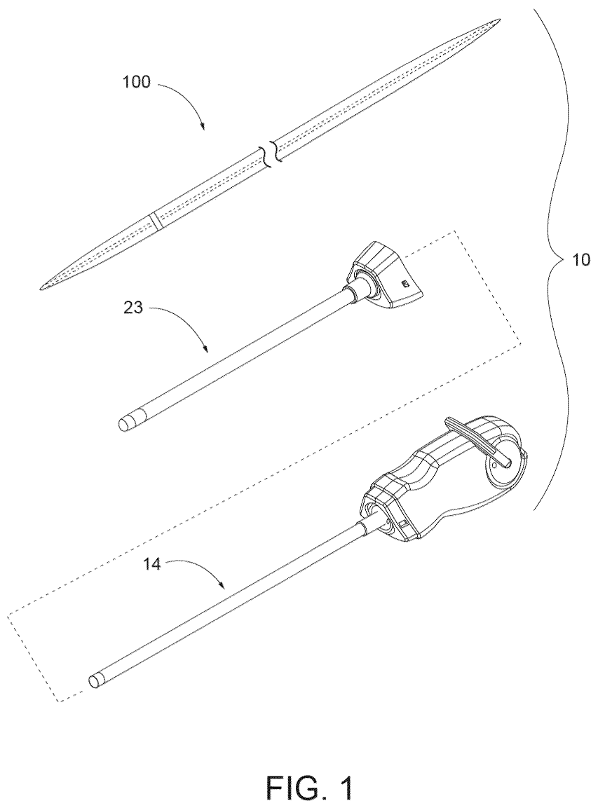

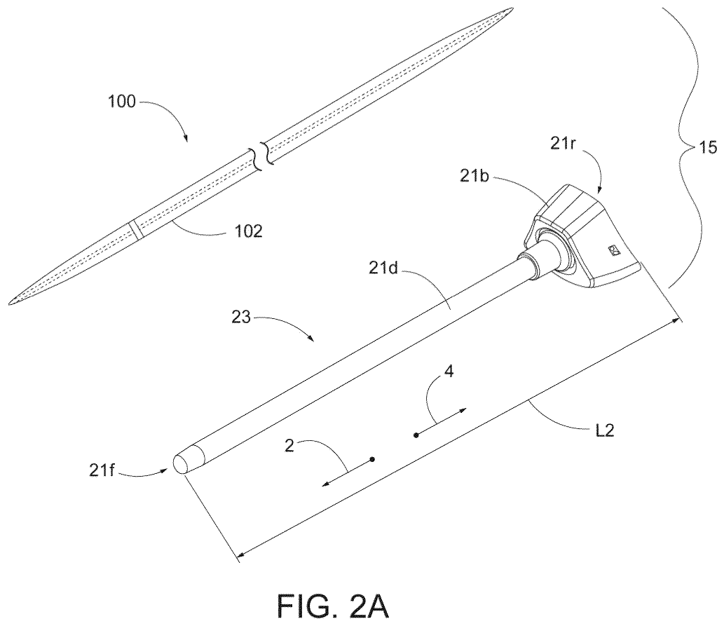

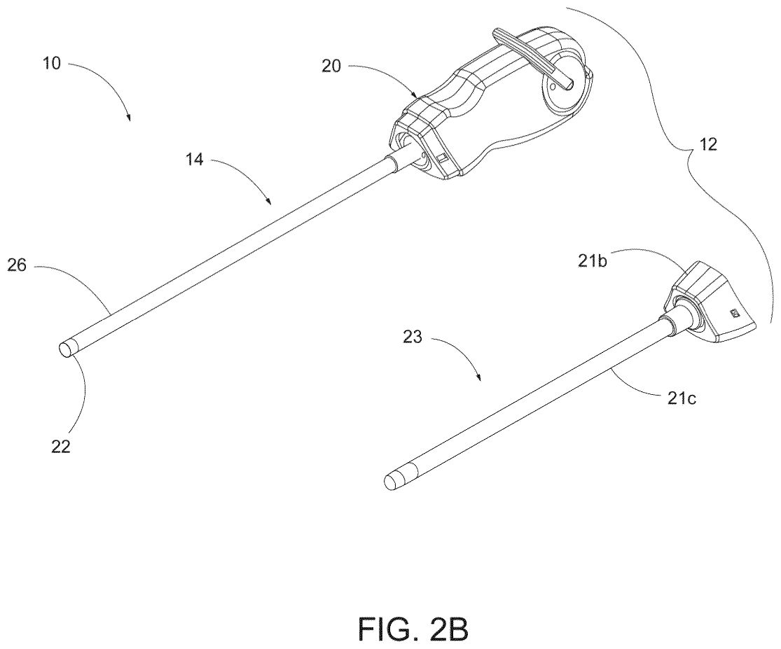

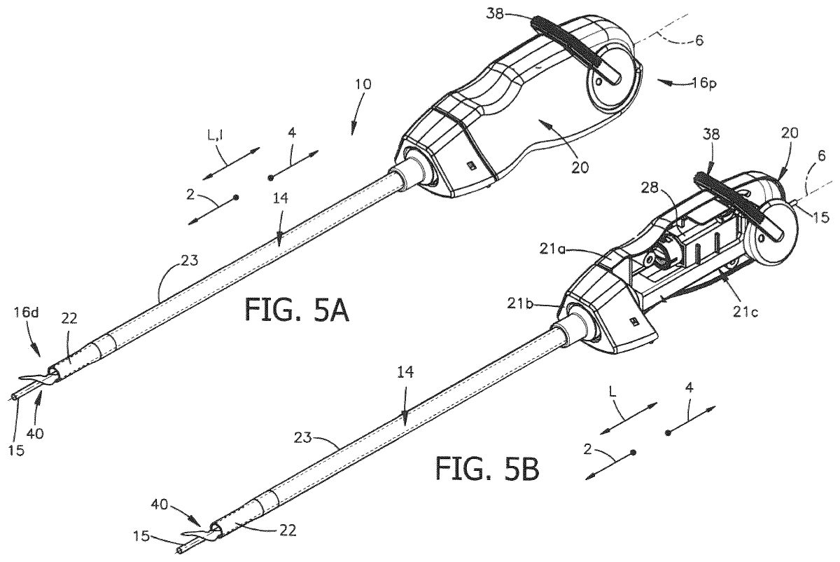

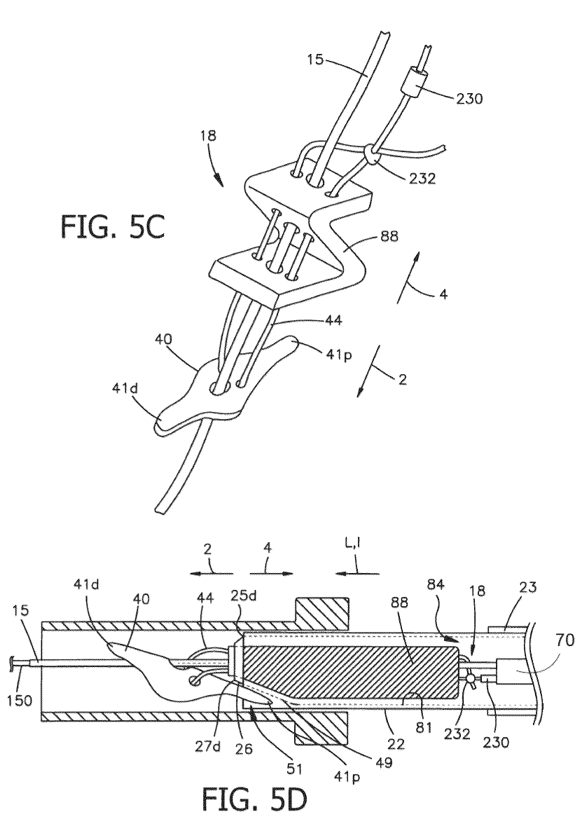

Referring to FIGS. 1-2B, the vascular closure system 10 includes an introducer 100 and a closure device 12 that is configured to seal the puncture in a vessel wall. The introducer 100 is configured to facilitate placement of the closure device 12 into the desired position within a puncture site of a vessel wall following a surgical procedure. The closure device 12 includes a deployment assembly 14 and an access sheath 23. The access sheath 23 can be inserted into the vessel and the deployment assembly 23 can be inserted into the access sheath 23 to position a sealing device or implantable unit 18 (FIG. 5C) into the vessel. The access sheath 23 and introducer 100 can be referred to as insertion assembly 15.

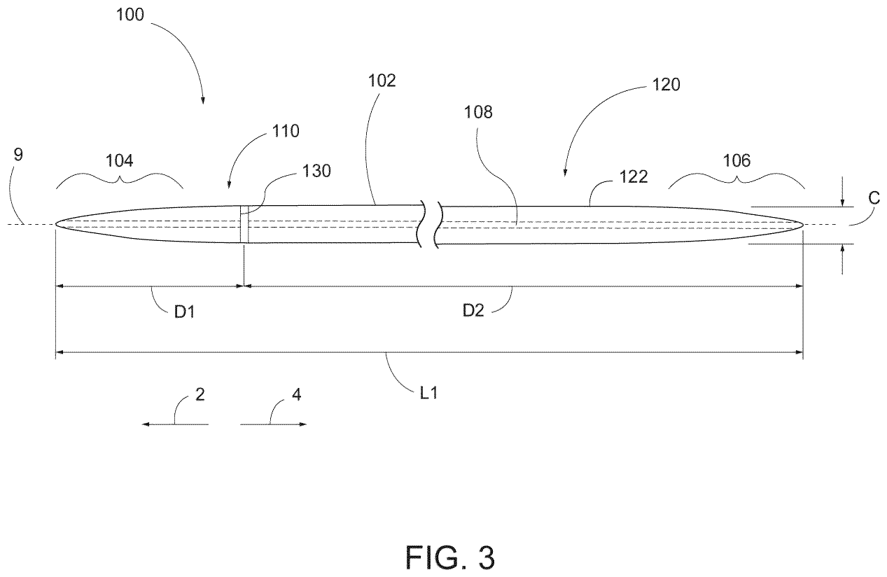

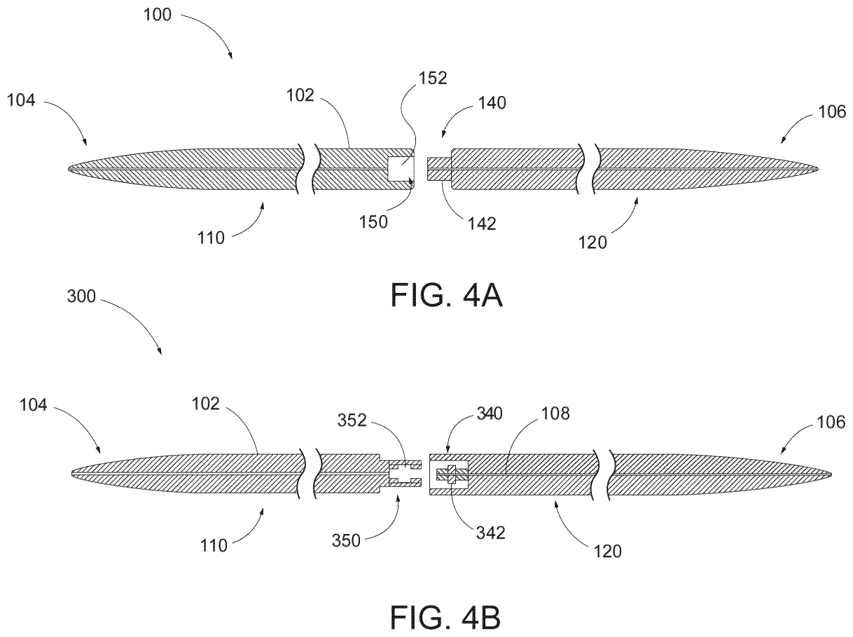

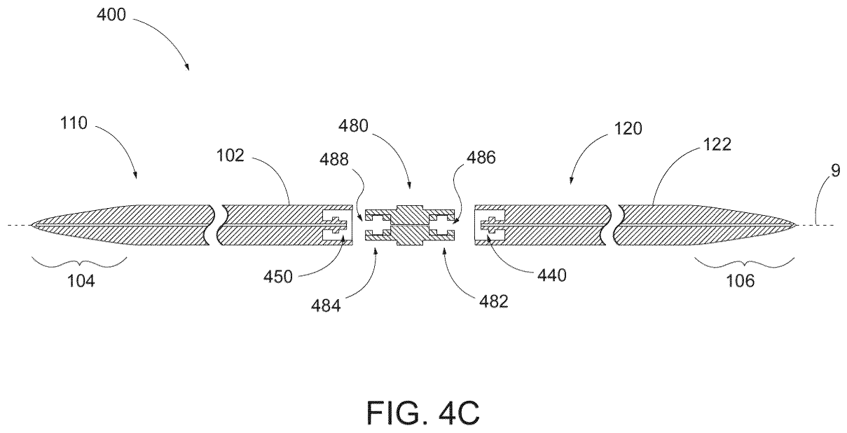

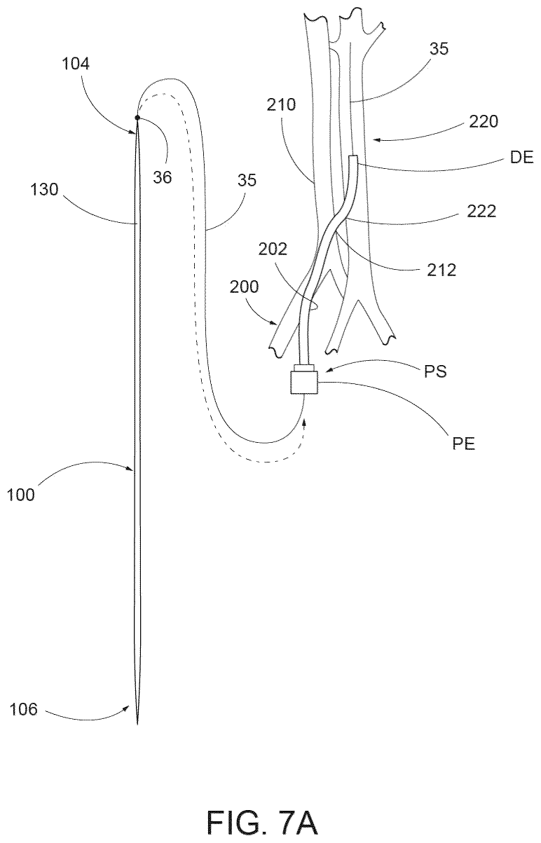

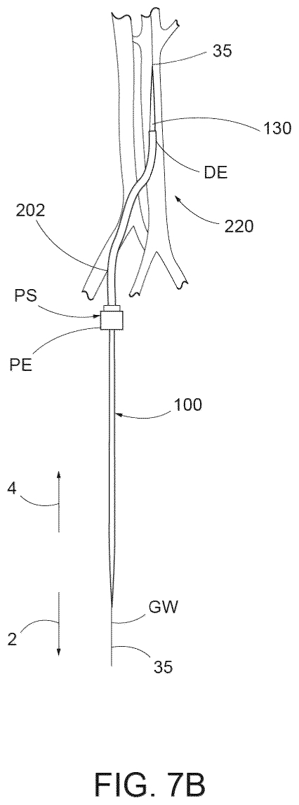

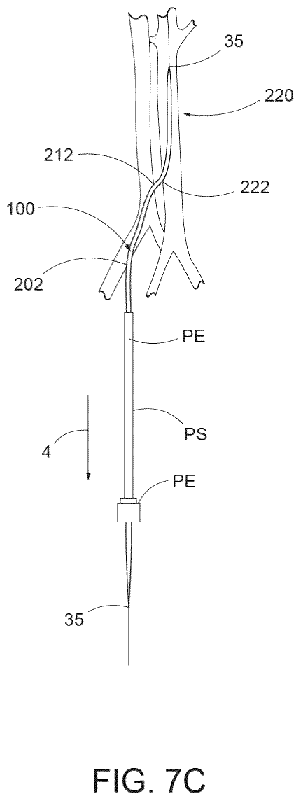

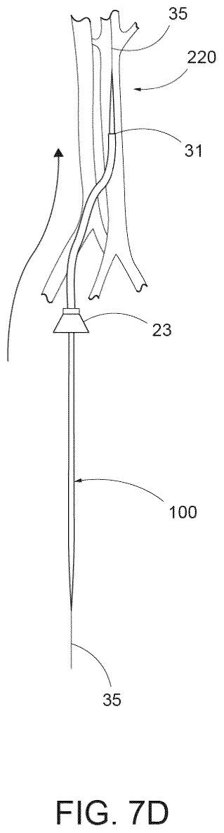

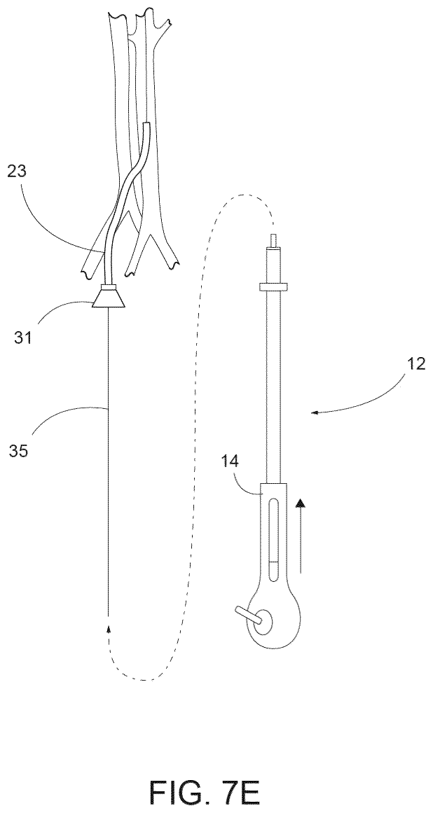

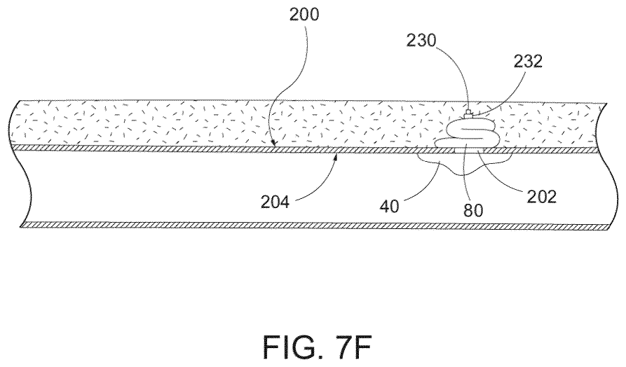

FIGS. 3-4B illustrates the embodiments of an introducer 100. The introducer 100 is configured to be inserted through the puncture along a guidewire 35 (FIG. 7A) that extends through the puncture into the vessel. The introducer 100 includes an introducer body 102 that is elongate along a central axis 9, a proximal tapered tip 106 a distal tapered tip 104 that is opposite to the proximal tapered tip 106 along the central axis 9, and a bore 108 that extends from the proximal tapered tip 106 to the distal tapered tip 104 along the central axis 9. The bore is sized to receive the guidewire 35 (not shown) therethrough. The distal tapered tip of the introducer is configured to be inserted into the vessel along the guidewire 35.

In one embodiment as shown in FIGS. 4A and 4B, the introducer 100 includes a proximal component 120 that defines the proximal tapered tip 106, and a distal component 110 that defines the distal tapered tip 104. The proximal component 120 and the distal component 110 are configured to be coupled together to define the introducer body 102 of the introducer. The proximal component defines a proximal portion of the bore 108. The distal component defines a distal portion of the bore 108 that is aligned with the proximal portion of the bore when the proximal and distal components are coupled together.

The introducer body 102 defines an outer surface 122 and outer cross-sectional dimension C that is perpendicular to the central axis 9. As illustrated, the outer cross-sectional dimension C does not vary along an entirety of the introducer body 102 between the proximal tapered tip 106 and the distal tapered tip 104. The proximal tapered tip 106 tapers from the outer surface 122 toward the central axis 9 along a proximal direction 4 away from the distal tapered tip 104. Furthermore, the distal tapered tip 104 tapers from the outer surface 122 toward the central axis 9 along a distal direction 2 that is opposite to the proximal direction 4. The introducer 100 (or introducer body 102) is configured to be inserted through the front end 21f and into the lumen of access sheath 23 such that a movable interference fit is attained between the access sheath 23 and the introducer 100. A moveable interference fit is where the introducer can be inserted into the access sheath 23 so as to permit the introducer 100 to move through the access sheath but does it not prevent its effective use. A user can therefore exert some level of force to advance the introducer along the guidewire into and partially through the access sheath 23 while still prevent free passage of blood or other fluids between the introducer 100 and the access sheath 23. Likewise, the access sheath 23 can be removed from the introducer 100 as needed. Thus, an interference fit interferes with free passage of blood or other fluids between the access sheath and introducer. However, a person of ordinary skill would understand that there may be some small amount of fluid loss even with such an interference fit.

As shown in FIGS. 2A and 3, the introducer body 102 further defines an introducer length L1 that extends from the proximal tapered tip 160 to the distal tapered tip 104 along the central axis 9. The access sheath 23 defines a sheath length L2 that extends from the front end 21f to the rear end 21r. The length L1 of the introducer 100 is at least two times longer than the length L2 of the sheath 23. In one embodiment of the present disclosure, such when the system is used to seal puncture deep in thoracic cavity following a trans-caval procedure, the introducer length L1 is between about 120 cm and about 130 cm. In one example of such an embodiment, the introducer length is between about 123 cm and about 127 cm. In another example, the introducer length is about 125 cm. In another embodiment, such as when the system is used to seal puncture in vessel within patient's limb, the introducer length L1 can be between about 20 cm and about 30 cm. In one example of such an embodiment, the introducer length is between about 23 cm and about 27 cm. In another such example, the introducer length is about 25 cm.

As shown in FIG. 3, the introducer body 102 includes at least one marker 130. Thus, there may be a single marker 130 or a plurality of markers. In an event, the marker 130 can be positioned to aid in identifying the location of the introducer 100 in the vessel. The marker 130 is one of a radio opaque band, a radio opaque ink, or a radio opaque paint. In accordance with one embodiment, a distance D1 from the distal tapered tip 104 to the marker 130 is less than a distance D2 from the marker 130 to the proximal tapered tip 106.

Referring to FIGS. 3-4C, the proximal component 120 and the distal component 110 are configured to be coupled together. In accordance with the illustrated embodiment, the proximal component 120 defines a first engagement member 140 opposite to the proximal tapered tip 106, and the distal component 110 defines a second engagement member 150 opposite to the distal tapered tip. The first engagement member 140 is configured to engage the second engagement member 150 so as to couple the proximal and distal components together.

The proximal component 120 and the distal component 110 are configured to be coupled together via one an interference fit. Accordingly, as illustrated in FIG. 4A, the first engagement member 140 defines a projection 142, and the second engagement member 150 defines a cavity 152 sized to receive the projection 142. However, it should be appreciated that one of the first engagement member 140 and the second engagement member 150 defines a cavity, and the other of the first engagement member 140 and the second engagement member 150 defines a projection that is sized to fit in the cavity. Accordingly, either component 110, 120 may have the projection or cavity as needed.

FIG. 4B illustrates another embodiment an introducer 300 that couples the proximal component and the distal component via a snap-fit connection. The introduce 300 is substantially similar to the introducer 100 described above and shown in FIG. 4A. For this reason, similar reference numbers are used to identify features that are common to introducer 100 and introducer 300. The introducer 300 includes a proximal component 120 and a distal component 110. In accordance with the embodiment shown in FIG. 4B, a first engagement member 340 includes a ridge 342 and the second engagement member 350 includes a groove 352 that is sized to receive the ridge 342. However, it should be appreciated that one of the first engagement member 340 and the second engagement member 350 defines a ridge, and the other of the first engagement member 340 and the second engagement member 350 defines a groove that is sized to receive the ridge.

In another alternative embodiment, the proximal component and the distal component an introducer may be configured to be coupled together via a threaded connection (not shown). For instance, one of the first engagement member and the second engagement member define external threads, and the other of the first engagement member and the second engagement member define internal threads configured to threadably mate with the external threads.

FIG. 4C illustrates another embodiment an introducer 400 that couples the proximal component and the distal component via a coupler 480. The introducer 400 is substantially similar to the introducer 100 described above and shown in FIG. 4A. For this reason, similar reference numbers are used to identify features that are common to introducer 100 and introducer 400. As shown in FIG. 4C, the introducer 400 includes a coupler 480 having a first end 482 and a second end 484 opposite the first end 482. The first end 482 is configured to be coupled to the proximal component 120 and the second end 484 is configured to be coupled to the distal component 110. The first and second ends 482 and 484 have first and coupling members 486 and 488, respectively. The first coupling member 486 can connect to the first engagement member 440 of the proximal component 120. The second coupling member 488 can connect to the second engagement member 450 of the distal component 110. The proximal component 120, the coupler 480, and the distal component 110 when coupled together can define the introducer 400. The engagement members 440, 450 and the coupling members 486,488 can be ridges, projections, tabs, or other structures or devices that can coupled parts to each other. Accordingly, the coupler 380 and ends of the distal component 110 and proximal component 120 may be configured for any type of connection, such as an interference fit, snap-fit, threaded connection, or the like.

Referring generally to FIGS. 3-4C, the proximal tapered tip 106 of the introducer 100 is configured to be inserted into the front end 21f and of the access sheath 23 and through the lumen of access sheath 23. The scaling element 18 extends out from the front end 21f of the access sheath when the portion of the vascular device is coupled to the hub 21b.

As shown in FIGS. 1 and 2A, the access sheath 23 is configured to be inserted over the introducer 100 and into the vessel. The access sheath 23 includes a hub 21b and shaft 21d that extends from the hub 21b in the distal direction 2. The access sheath 23 has a front end 21f, a rear end 21r opposite to the front end 21f, and a lumen (not numbered) that extends from the front end 21f to the rear end 21r. The rear end 21r of the access sheath includes the hub 21b that is configured to be coupled to a portion of the deployment assembly 14. When sheath 23 is coupled to the deployment assembly 14, the shaft 21d extends along the release component 22 and delivery component 26 in the distal direction 2.

Referring to FIG. 5A-5D, a vascular closure device 12 includes a sealing device or implantable unit 18 at least partially disposed within a deployment assembly 14. The vascular closure device 12 can be configured such that after a distal portion of deployment assembly 14 is inserted through a puncture site of the vessel, the sealing device 18 is deployed to thereby seal or otherwise close the puncture site of the vessel. The deployment assembly 14 is configured to control orientation of a toggle 40 of the sealing device 18 in an easier and more efficient manner during deployment of the sealing device 18. Furthermore, the deployment assembly 14 is configured to reduce forces required to deploy the sealing device 18 and seal the puncture.

In accordance with the illustrated embodiment, the deployment assembly 14 includes a release component 22 that restrains the toggle 40, a delivery component 26 that contains at least a portion of the toggle 40 and a suture 44 of the scaling device 18, a guide member 35, and one or more actuators 38 coupled to the release component 22. The deployment assembly 14 may also include a tamper 70, in the form a tube, that extend along the suture 44 is a located proximal with respect to the locking member 230 (See FIG. 5D). The guide member 35 extends through the scaling device 18 and is configured to receive a guidewire 35 as will be discussed below. In another example, the deployment assembly 14 can be configured so that the guidewire 35 extends along the side of the toggle 40. The release component 22 is operatively associated with the suture 44 such that actuation of the actuator 38 causes the release component 22 to 1) release the toggle 40, and 2) apply tension to the suture 44, which urges the toggle 40 against the delivery component 26 and orients the toggle 40 in the sealing position. The guide member 35 is configured to be removed from at least the scaling device 18 prior the sealing device 18 scaling the puncture.

Turning to FIG. 5C, the sealing device 18 includes the toggle 40 connected to the suture 44, a plug 88 coupled to the suture 44 and spaced from the toggle 40 in a proximal direction 4, and a locking member 230 proximal to the plug 88. The toggle 40 includes a distal end 45d and a proximal end 41p opposite to the proximal end 41p, and a plurality of apertures (not numbered) extending therethrough. The suture 44 extends through the apertures as illustrated such that an end of the suture 44 is formed into a slidable knot 232. The knot 232 is slidable along the suture 44 between the plug 88 and the locking member 230. In an implanted state, the toggle 40 is adjacent an inner surface of the vessel and the locking member 230 squeezes the toggle 40 the plug 88 against the vessel to seal the puncture. See for example FIG. 7F.

The scaling device 18 is formed with materials suitable for surgical procedures such as any biocompatible material. For example, the toggle 40 can be made of a polylactic-coglycolic acid or other synthetic absorbable polymer that degrades in the presence of water into naturally occurring metabolites. In other embodiments, the toggle can be made of stainless steel, biocorrodible iron, and biocorrodible magnesium. It should be appreciated, however, that the toggle 40 can be made of other materials and can have other configurations so long as it can be seated inside the vessel against the vessel wall. The plug 88 can comprise a strip of compressible, resorbable, collagen foam and can be made of a fibrous collagen mix of insoluble and soluble collagen that is cross linked for strength. It should be appreciated, however, that the plug member 88 can have any configuration as desired and can be made from any material as desired. The suture 44 can be any elongate member, such as, for example a filament, thread, or braid.

Referring again FIGS. 5A, 5B and 5D, the deployment assembly 14 is elongate along a longitudinal direction L and includes a rear end 16p and a front end 16d spaced from the rear end 16p along an axis 6 that is aligned with the longitudinal direction L. The longitudinal direction L can include and define a distal direction 2 that extends from the rear end 16p toward the front end 16d. Further, the longitudinal direction L can include and define a proximal direction 4 that is opposite the distal direction 2 and that extends from front end 16d toward the rear end 16p. The deployment assembly 14 is configured to insert the toggle 40 into the vessel along an insertion direction I (see FIG. 4). The longitudinal direction L can be aligned with the insertion direction I during a portion of the scaling procedure.

Turning to FIGS. 5A and 5B, in accordance with the illustrated embodiment, the deployment assembly 14 includes a handle member 20, the release component 22 supported by the handle manner 20 and extending from handle member 20 in the distal direction 2, the delivery component 26 also supported by the handle member 20 and extending along the distal direction 2, and a tensioner 28 supported by the handle member 20 and positioned adjacent to the release component 22. A portion of delivery component 26 is shown in dashed lines in FIGS. 5A and 5B.

The actuator 38 is coupled to both the handle member 20 and the release component 22. As noted above the actuator 38 is configured to 1) cause the release component 22 to move in the proximal direction 4 from a first or initial position relative to the delivery component 26 into a second or releasing position relative to the delivery component 26, and 2) apply a tensile force to the suture 44 during or subsequent to movement of the release component 22 from the initial position into the released position. The description below refers to the release component 22 being moveable relative to the delivery component 26. But the deployment assembly 14 can be configured so that the delivery component 26 is moveable relative to the release component 22. The deployment assembly 14 also includes the guide member 35 that extends through the deployment assembly 14, and an outer sheath 23 that contains and supports portions of the release component 22 and delivery component 26. Furthermore, the actuator 38 may be adapted to operate, or cause move the tamper 70 along the suture 44 to tamp the sealing unit into a tamped, deployed configuration. In alternative embodiment, an separate actuator may be used to control the tamper 70.

Continuing with FIGS. 5A and 5B, the handle member 20 includes a housing 21a and a cavity 21c defined at least partly by housing 21a and a nose 21b of the access sheath 23. The cavity 21c is sized to contain a portion of the release component 22, delivery component 26, and the tensioner 28.

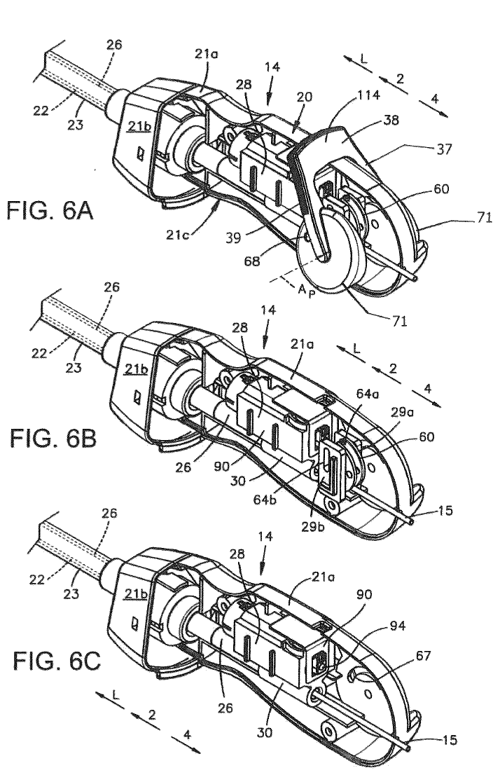

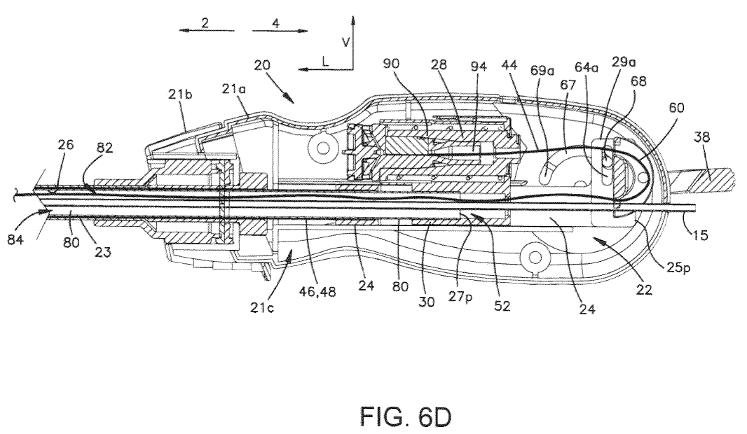

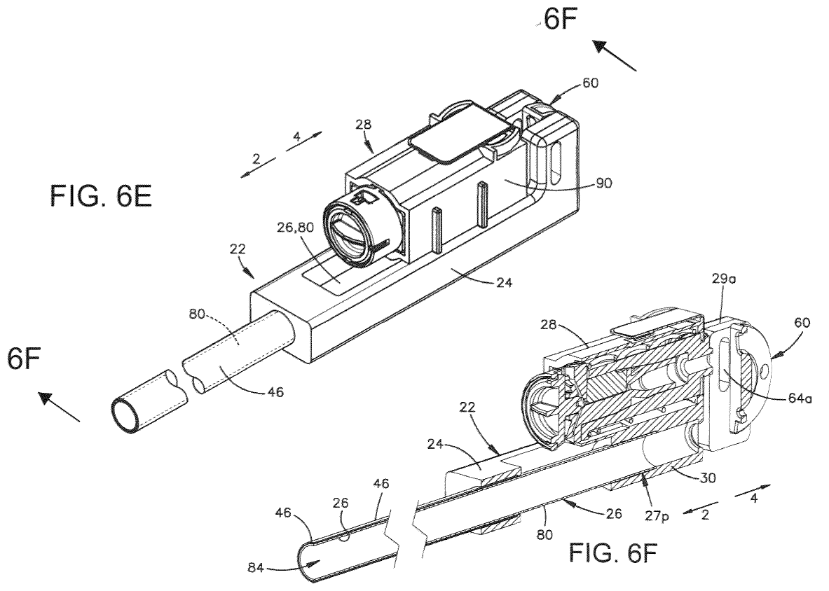

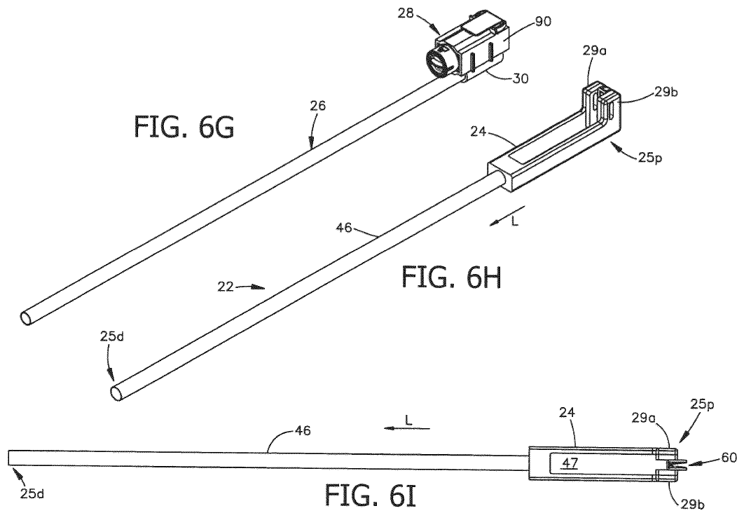

Turning to FIGS. 5B, 6H and 6I, the release component 22 is elongate along a first or longitudinal direction L defines a distal end 25d and a proximal end 25p spaced from the distal end 25d along the longitudinal direction L. In accordance with the illustrated embodiment, the release component 22 includes a release hub 24 and a release tube 46 that is fixed to the release hub 24 extends from the release hub 24 in the distal direction 2. The release hub 24 includes a pair of tabs 29a, 29b disposed at the proximal end 25p of the release component 22. A pulley 60 is coupled to the tabs 29a, 29b and defines a curved track that receives the suture 44 as will be explained below. The hub 24 defines a slot 47 that is elongate along the longitudinal direction L and is aligned with the release tube 46. The slot 47 is sized to receiver a coupler 30 of the tensioner 28.

The release tube 46 includes a release tube body 48 that is elongate along the longitudinal direction L. The release tube body 48 defines a release tube channel 52 that extends along the longitudinal direction L from the hub 24 toward the proximal end 25p. In the illustrated embodiment, the release tube channel 52 (FIG. 6D) extends completely through the release tube body 48 from the hub 24 to the distal end 25d. Furthermore, in the illustrated embodiment the release tube body 48 is cylindrical such that the release tube channel 52 is radially enclosed. It should be appreciated, however, that the release tube channel 52 can extend partially through the release tube body 48 as desired and that the release tube body 48 can have other configurations as desired. For example, the release tube body 48 can be U-shaped such that the release tube channel 52 is partially radially open. As shown, the release tube channel 52 is sized to slidably receive a portion of the delivery component 26 such that the release component 22 is movable relative to the delivery component 26.

Referring to FIGS. 6A, 6B, 6D, and 6I, the pulley 60 is disposed at the proximal end 25p of the release component 22. As shown, the suture 44 extends around the pulley 60 along the guide track and into the tensioner 28. As the release component 22 is pulled in the proximal direction 4, the pulley 60 pulls the suture 44 in proximal direction 4 thereby applying a tensile force to the toggle 40. In such an embodiment, the tensioner 28 is positioned alongside the release component 22. It should be appreciated, however, that in some embodiments, the tensioner 28 is positioned proximal to the release tube and is in-line with the release component 22 such that the suture 44 extends through the release tube and into the tensioner 28 along the first direction L.

With continued reference to 6A, 6B, 6D, and 6I, the release component 22 can include at least one mating member 64 that mates with a corresponding mating member 68 of the actuator 38 to thereby transfer the motion of the actuator 38 to the release component 22. In the illustrated embodiment, the release component mating member 64 is a pair of slots 65a and 65b defined by the respective pair of tabs 29a and 29b. Each slot 65a and 65b is elongate along a direction a vertical direction V that is perpendicular to the first direction L. The actuator 38 mating member 68 can be operatively engaged with elongate slots 65a and 65b of release component 22 such that actuation of the actuator 38 causes the release component 22 to translate along the first direction L. It should be appreciated, however, that the mating member 64 can have any configuration as desired. For example, the mating member 64 can be a bore having a diameter that is equal to that of the pin such that translation of the actuator 38 along the first direction L causes the release component 22 to translate along the first direction L.

As shown in FIGS. 5B, 6D-6G, the delivery component 26 is coupled to the tensioner 28 and extends along the release component 22 toward the front end 16d of the deployment assembly 14. In accordance with the illustrated embodiment, because the tensioner 28 is fixed to the housing 21a, the delivery component 26 is fixed to the housing 21a and thus the handle member 20. The delivery component 26 includes a delivery tube body 80 that is elongate along the first direction L and defines a distal end 27d and a proximal end 27p spaced from the distal end 27d in the first direction L. The delivery tube body 80 defines an inner surface 81, which in turns defines a delivery tube channel 84 that extends at least partially through the delivery tube body 80 along the first direction L. As illustrated embodiment, the delivery tube channel 84 extends completely through the delivery tube body 80 from the proximal end 27p to the distal end 27d. However, the channel 84 can extend along a portion of the delivery tube body 80. Furthermore, in the illustrated embodiment the delivery tube body 80 is cylindrical such that the delivery tube channel 84 is radially enclosed. It should be appreciated, however, that the delivery tube channel 84 can extend partially through the delivery tube body 80 as desired and that the delivery tube body 80 can have other configurations as desired. For example, the delivery tube body 80 can be U-shaped such that the delivery tube channel 84 is partially radially open. As illustrated, the proximal end 27p of delivery component is fixed to the tensioner 28 and the distal end 27d of delivery component is configured to hold at least a portion of the sealing device 18 (FIG. 5D).

The delivery tube channel 84 is sized to retain at least a portion of the sealing device 18. In particular, the plug 88 and locking member 230 are retained within the delivery tube channel 84, while the toggle 40 is configured to be initially trapped between the delivery component 26 and the release component 22. For instance, the distal end 25d of the release tube 48 defines an offset surface 49, which can be angled with respect to the longitudinal axis 6. The offset surface 49 and inner surface 81 of the delivery tube 80 define a cavity 51 that receives the proximal end 41p of the toggle 40 when release component 22 is in the initial position (as shown in FIG. 5D). The angle of the offset surface 49 can define the orientation of the toggle 40 in this initial position, whereby the distal end 45D of the toggle 40 is spaced some distance in the distal direction 2 beyond the distal ends 25d and 27d of the release component 22 and delivery component 26, respectively. The suture 44 extends from the toggle 40 through the delivery tube channel 84, through the proximal end 27p (FIG. 6D) around the pulley 60 along the guide track and is coupled to the tensioner 28. The guide member 35 extends through the channel 84 and exits the front end 16d of the vascular closure device 12. When the actuator 38 is actuated as will be further detailed below, the release component 22 moves in the proximal direction 4 thereby releasing the proximal end 41p of the toggle 40 from between the release component 22 and the delivery component 26. As the release component 22 moves in the proximal direction 4, the suture 44 will be pulled in the proximal direction 4 to thereby place the suture 44 in tension and urge the toggle 40 against the distal end 27d of the delivery component 26. At this point, the toggle 40 is oriented in the scaling position (see FIG. 6E). In the scaling position, the toggle 40 has been repositioned so that the toggle 40 is placed against the distal end 27d of the delivery component 26 and is oriented more transversely with respect to the axis 6 compared to the position when the toggle 40 is restrained by the release component 22.

As shown in FIGS. 6D-6G, the tensioner 28 disposed on the delivery component 26 and is positioned alongside the release component 22 so as to receive the suture 44 as noted above. In accordance with the illustrated embodiment, the tensioner 28 includes a tensioner housing 90, a coupler 30 that extends from the housing 90 and is attached to the delivery component 26, and a drag member 94 disposed within the tensioner housing 90. The suture 44 extends into the tensioner housing 90 through the drag member 94 and such that a frictional force is applied to the suture 44 by the drag member 94. The tensioner housing 90 is coupled the housing 21a and fixed thereto. The coupler 30 as illustrated is a tubular component that receives the proximal end 27p of the delivery tube body 80. As illustrated, the delivery tube body 80 is fixed to the coupler 30 which indirectly fixes the delivery component 26 to the housing 21A.

The suture 44 extends from the proximal end 27p of the delivery tube body 80, through the coupler 30, around the pulley 60 and into the drag member 94 and is spooled within the tensioner housing 90 (not shown). Spooling the suture 44 in tensions housing 90 allows suture 44 to dispends from the deployment assembly 14 when the deployment assembly 14 is pulled is proximal direction 2 to thereby deploy the sealing device 18 (see FIGS. 6F and 6G). The frictional force applied to the suture 44 by the drag member 94 can be high enough to maintain the suture 44 in tension after the actuator 38 has been actuated and the toggle 40 has been urged against the distal end 27d of the delivery component 26. At the same time the frictional force applied to the suture 44 by the drag member 94 can be low enough to allow the suture 44 to dispense from the tensioner housing 90 when the deployment assembly 14 is pulled in a proximal direction 4 relative to the toggle 40. In the illustrated embodiment, the drag member 94 is a silicon member that pinches the suture 44. The tensioner 90 and drag member 94 can be similar the tensioner described in U.S. Patent Application Publication No. 2013/0178895. It should be appreciated, however, that the drag member 94 can have other configurations as desired.

Turning to FIGS. 6A-6D, the deployment assembly 14 can include one or more actuators that are configured to transition the release component 22 into to releasing position and to cause a tension to be applied to suture 44 when toggle 40 is released from the release component 22 as described above. As noted above, the actuator 38 can include the mating member 68 that operatively engages the mating member 64 of the release component 22 such that motion of the actuator 38 relative to the handle member 20 causes the release component to translate in the proximal direction 4 and further applies a tension to the filament.

In accordance with the illustrated embodiment, the actuator 38 can be configured as a lever that is rotatably coupled to the handle member 20. The actuator 38 or lever can include a pair of side members 71a and 71 rotatably coupled to each side of the housing 21a, a first leg 37a that extends from one of the side members 71a, a second leg 37b that extends from the other side member 71b, and a transverse member 39 that connects the first leg 37a to the second leg 37b. The actuator 38 is configured to pivot about a pivot axis A_{P} that is perpendicular to the axis 6. The pivot axis A_{P} may or may not intersect axis 6. The housing 21a defines a curved housing slot 67 that is curved with respect to the pivot axis A_{P}. The curved housing slot 67 has a first end 69a (FIG. 6D) and second end (not numbered) spaced apart from the first end along the proximal direction 4. The mating member 68 of the actuator 38 can be a pin 68 that is coupled to and extends between the side members 71 and 71b at a location that is offset from the pivot axis A_{P}. The pin 68 extends through curved housing slot 67 and through the elongate slots 64a and 64a of the hub 24 of the release component 22 such that the actuator 38 is operatively coupled to the release component 22.

In use, as the actuator 38 pivots about the pivot axis A_{P}, the pin 68 moves from the first end 69a the curved housing slot 67 toward the second end of the curved housing slot 67, and also moves along the slots 64a and 64b along the vertical direction V. Because the release component 22 is moveable relative to housing 21a, as pin 68 moves along the curved housing slot 67, the pin 68 advances the hub 24 of the release component 22 in the proximal direction 4. The result in accordance with the illustrated embodiment is that rotation of the actuator 38 causes the release component 22 to translate in the longitudinal direction L. It should be appreciated, however, that the actuator 38 can have other configurations as desired and is not limited to the disclosed lever.

In operation, the deployment assembly 14 is initially configured to insert the toggle 40 into the vessel. When the actuator 38 is actuated, the release component 22 moves in the proximal direction 4 relative to the delivery component 26 into the releasing position (not illustrated) thereby releasing the proximal end 41p of the toggle 40 from between the release component 22 and the delivery component 26. As the release component 22 moves in the proximal direction 4, the suture 44 will be pulled in the proximal direction 4 to thereby place the suture 44 in tension and urge the toggle 40 against the distal end 27d of the delivery component 26. At this point, the toggle 40 is oriented in the sealing position (see FIG. 6E). Accordingly, the release component 22 is configured to restrain the toggle 40 of the sealing device 18 during insertion of the vascular closure device 12 into the vessel and subsequently release the toggle 40 so that the toggle 40 can be oriented for the sealing procedure.

The release component 22 is also in communication the suture 44 via the pulley 60 such that when the actuator 38 is actuated the release component 22 pulls the suture 44 in the proximal direction to thereby place the suture 44 in tension. Application of tension along the suture 44 urges the toggle 40 against the distal end 27d of the delivery component 26 and orients the toggle 40 into the sealing position. In the illustrated embodiment, the actuator 38 and release component 22 are configured such that continuous movement of the actuator 38 relative to the housing 21a will move the release component 22 in the proximal direction 4, thereby releasing the toggle 40 from the release component 22 and subsequently apply tension to the suture 44. It should be appreciated, however, that in some embodiments the suture 44 can be tensioned as the toggle 40 is being released. It should further be appreciated that in some embodiments, the deployment assembly 14 can include a first actuator to release the toggle 40 and a second actuator that tensions the suture 44.

The release component 22 and delivery components 26 are described above has having tubular shaped bodies. It should be appreciated that the release and delivery components can have other configurations. For instance, the release component can be elongate rod, or an elongate rod with a tubular ring coupled to its distal end. The delivery component can be configured such that only a portion thereof has a tubular shape.

Embodiments of the present technology will now be described with respect to exemplary large bore procedures that utilize the vascular closure system 100 illustrated in FIGS. 7A-7F. In order to perform any of the related procedures, the user gains percutaneous access to, for example, the femoral artery, causing a puncture site in the artery. To gain percutaneous access to the artery, the Seldinger technique may be used. For example, a hollow bore needle is inserted into the vessel 200 through a procedure sheath PS (referred to as the first access sheath). A guidewire is then advanced through the hollow needle into the femoral artery a sufficient distance to allow removal of the needle without the guidewire pulling out of the vessel. Removing the needle leaves the guidewire in place, with a portion of the guidewire extending into the artery and proximal end PE of the sheath PS extending out patient. The guidewire, extending from outside the patient into the femoral artery, provides for an entry guide for other medical devices including the access sheath 23, introducer 100, and vascular closure device 12. Therefore, once the guidewire is positioned in the vessel of the patient, catheters, or introducers, of gradually increasing diameters are advanced over the guidewire and through the puncture into the artery to further open the puncture site. Then, a procedure access sheath set (i.e. an introducer 100 inside a procedure sheath PS) is moved along the guidewire such that a distal end DE of the sheath PS moves into the vessel through the puncture site. And once positioned, the introducer can be removed such that the sheath provides for sizable access to the vessel interior from outside the body. After the relevant procedure is completed, the puncture site in the artery created during percutaneous access of the artery may be closed. The vascular closure system may be used to seal the puncture site.

In some instances, however, access through the femoral artery as described above is not indicated due to condition of the vessel between the femoral artery and the aorta. In such cases, a trans-caval procedure can be used to access the aorta. As shown in FIG. 7A, the trans-caval procedure includes guiding a guidewire 35 through into through a first puncture 202 in a femoral vein 200 and further into and a portion of the inferior vena cava 210. The method include creating a second puncture 212 in the portion of the inferior vena cava 210 and creating a third puncture 222 in a femoral artery 220 and a portion of the aorta 220. The punctures can formed with a tip of the guidewire 35, such as by burning. Next, the distal end DE of the procedure access sheath PS is guided along the guidewire 35 through the second and third punctures. When the sheath PS is in place, a medical device, such as a catheter, is inserted through the first access sheath PS. When the procedure is completed, the catheter is removed from the procedure access sheath PS and the guidewire 35.

Continuing with FIG. 7A, a method includes positioning a tapered distal end 104 of an introducer over a proximal end 36 of a guidewire 35 that extends through a puncture 202 in a vessel 200, e.g. a vena cava, such that the guidewire 35 enters a bore 108 of the introducer. In the example illustrated, the procedure sheath PS and guidewire 35 extends from the outside the patient into the femoral venal cava.

Next, as shown in FIG. 7B, the method includes advancing the introducer 100 along the guidewire 35 in a distal direction 4 so that the tapered distal end enters the proximal end PE of the first access sheath PS. The introducer 100 further advanced out of a distal end DE of the access sheath PS that is spaced from the proximal end PE of the access sheath PS in the distal direction 4. The introducer 100 is advanced in the distal direction until a marker 130 disposed toward the tapered distal end is positioned within a predetermined distance of the puncture of the vessel.

As shown in FIGS. 7B and 7C, the method includes removing the access sheath PS from the punctures 202, 212 and 214 while maintaining a portion of the tapered distal end 104 of the introducer 100 in the aorta 220 (or some other vessel as the case may be).

As shown in FIG. 7D, after the removing step, the procedure includes the step of inserting the access sheath 23 of system 10 (also referred to as the second access sheath) over the tapered proximal end of the introducer until a distal or front end 31 of the access sheath 23 extends through the puncture of the vessel.

As shown in FIG. 7D, the method includes the step of removing the introducer 100 from the access sheath 23 and the guidewire 35. The exchange of sheaths PS and 23 limits blood loss and ensure smooth transition between the interventional procedure and sealing the puncture site.

As shown in FIG. 7E, the method includes advancing a vascular closure device, for instance the deployment assembly 14, into the access sheath 23 to seal the puncture. In one example, the method can also include the steps of sealing the puncture 222, sealing puncture 212 and sealing puncture 202. In most cases, the sealing device 18 is deployed as illustrated in FIG. 7F. As deployed, the toggle 40 is adjacent the vessel wall 204, the plug 88 is collapses against the outer surface of the wall 204 and opposite the toggle 40. The knot 232 and lock member 230 secure the plug 88 in place, compressing the plug 88 and toggle 40 together.

It should be appreciated that the introducer 100 can be assembled during manufacture or at the surgical site prior to its use as described above. The method can include coupling a distal component 110 of the introducer 100 to a proximal component 120 of the introducer, wherein the distal component 110 defines the tapered distal end 104 and the proximal component 120 defines the tapered proximal end 106.

The method described above is related to a trans-caval procedure. It should be appreciated that the vascular closure system can be used to seal punctures in a femoral artery. In particular, the vascular closure system may be used to see so-call large bore punctures, such as 10F French, 12 French, 14 French or larger sized bore. Such a system is typically used to seal a puncture in vessel within patient's limb.

While the foregoing description and drawings represent the preferred embodiment of the present invention, it will be understood that various additions, modifications, combinations and/or substitutions may be made therein without departing from the spirit and scope of the present disclosure as defined in the accompanying claims. In particular, it will be clear to those skilled in the art that the present disclosure may be embodied in other specific forms, structures, arrangements, proportions, and with other elements, materials, and components, without departing from the spirit or essential characteristics thereof. One skilled in the art will appreciate that the present disclosure may be used with many modifications of structure, arrangement, proportions, materials, and components, which are particularly adapted to specific environments and operative requirements without departing from the principles of the present disclosure. In addition, features described herein may be used singularly or in combination with other features. For example, features described in connection with one component may be used and/or interchanged with features described in another component. The presently disclosed embodiment is therefore to be considered in all respects as illustrative and not restrictive, the scope of the present disclosure being indicated by the appended claims, and not limited to the foregoing description. It will be appreciated by those skilled in the art that various modifications and alterations of the present disclosure can be made without departing from the broad scope of the appended claims. Some of these have been discussed above and others will be apparent to those skilled in the art.

We claim:

1. A method, comprising:
inserting an introducer over a guidewire and into a first access sheath in a distal direction until a distal end of the introducer is positioned through a puncture in a vessel;
removing the first access sheath from the puncture while maintaining the distal end of the introducer and a distal end of the guidewire in the vessel;
passing a second access sheath including a hub over the introducer and the guidewire in the distal direction until a distal end of the second access sheath extends through the puncture into the vessel such that an interference fit allowing relative movement between the second access sheath and the introducer is attained after the second access sheath is over the introducer, wherein the interference fit allows the introducer to 1) advance along the guidewire into and partially through the second access sheath and 2) move relative to the second access sheath after the second access sheath is over the introducer and while coupled to the hub and simultaneously inhibiting fluid from passing between the second access sheath and the introducer; and
removing the introducer while leaving the second access sheath in place.

2. The method of claim 1, further comprising advancing a vascular closure device into the second access sheath to seal the puncture.

3. The method of claim 2, wherein advancing the vascular closure device into the second access sheath to seal the puncture further comprises advancing the vascular closure device into the second access sheath along the guidewire.

4. The method of claim 1, wherein the distal end of the introducer is tapered, a proximal end of the introducer is tapered, and a portion extending from the distal end-to the proximal end of the introducer has a substantially constant cross-sectional dimension.

5. The method of claim 1, wherein the introducer defines an introducer length that extends from a proximal end to the distal end along a central axis, wherein the introducer length is between about 120 cm and about 130 cm, inclusive.

6. The method of claim 5, wherein the introducer length is between about 123 cm and about 127 cm, inclusive.

7. The method of claim 1, wherein the introducer defines an introducer length that extends from a proximal end to the distal end along a central axis, wherein the introducer length is between about 20 cm and about 30 cm, inclusive.

8. The method of claim 7, wherein the introducer length is between about 23 cm and about 27 cm, inclusive.

9. The method of claim 1, wherein the introducer includes at least one marker.

10. The method of claim 9, wherein the at least one marker is one of a radio opaque band, a radio opaque ink, or a radio opaque paint.

11. The method of claim 1, wherein the interference fit inhibits passage of blood or other fluids between an inner surface of the second access sheath and an outer surface of the introducer.

12. A method, comprising:
inserting a distal tapered end of an introducer over a guidewire and into a first access sheath in a distal direction until the distal tapered end of the introducer is positioned through a puncture in a vessel;
removing the first access sheath from the puncture while maintaining the distal end of the introducer and a distal end of the guidewire in the vessel;
passing a second access sheath over a proximal tapered end of the introducer and the guidewire in the distal direction until a distal end of the second access sheath extends through the puncture into the vessel, thereby allowing relative movement between the second access sheath and the introducer at any point after the access sheath is over the introducer and simultaneously inhibiting fluid from passing between the second access sheath and the introducer; and
removing the introducer while leaving the second access sheath in place.

13. The method of claim 12, further comprising advancing a vascular closure device into the second access sheath to seal the puncture.

14. The method of claim 13, wherein advancing the vascular closure device into the second access sheath to seal the puncture further comprises advancing the vascular closure device into the second access sheath along the guidewire.

15. The method of claim 12, wherein a portion of the introducer extending from the distal tapered end to the proximal tapered end has a substantially constant cross-sectional dimension.

16. The method of claim 12, wherein the introducer defines an introducer length that extends from the proximal tapered end to the distal tapered end along a central axis, wherein the introducer length is between about 120 cm and about 130 cm, inclusive.

17. The method of claim 16, wherein the introducer length is between about 123 cm and about 127 cm, inclusive.

18. The method of claim 12, wherein the introducer defines an introducer length that extends from the proximal tapered end to the distal tapered end along a central axis, wherein the introducer length is between about 20 cm and about 30 cm, inclusive.

19. The method of claim 18, wherein the introducer length is between about 23 cm and about 27 cm, inclusive.

20. The method of claim 12, wherein the introducer includes at least one marker.

21. A method, comprising:
inserting a distal tapered end of an introducer over a guidewire and into a first access sheath in a distal direction until the distal tapered end of the introducer is positioned through a puncture in a vessel;
removing the first access sheath from the puncture while maintaining the distal end of the introducer and a distal end of the guidewire in the vessel;
passing a second access sheath over a proximal tapered end of the introducer and the guidewire in the distal direction until a distal end of the second access sheath extends through the puncture into the vessel to form an interference fit with the introducer, wherein movement between the second access sheath and the introducer is not inhibited after the second access sheath is over the introducer, and the interference fit inhibits fluid from passing between the second access sheath and the introducer; and
removing the introducer while leaving the second access sheath in place.

22. The method of claim 21, further comprising advancing a vascular closure device into the second access sheath to seal the puncture.

23. The method of claim 21, wherein advancing the vascular closure device into the second access sheath to seal the puncture further comprises advancing the vascular closure device into the second access sheath along the guidewire.

24. The method of claim 21, wherein the distal end of the introducer is tapered, a proximal end of the introducer is tapered, and a portion extending from the distal end-to the proximal end of the introducer has a substantially constant cross-sectional dimension.

25. The method of claim 21, wherein the introducer includes at least one marker.

26. The method of claim 25, wherein the at least one marker is one of a radio opaque band, a radio opaque ink, or a radio opaque paint.
